(19) United States
(12) Patent Application Publication
(10) Pub. No.: US 2025/0387054 A1
(43) Pub. Date: Dec. 25, 2025

(54) PREVENTING YELLOWING AND FLUORESCENCE IN EPOXIDE DIAMINE NETWORKS

(71) Applicant: Senseonics, Incorporated, Germantown, MD (US)

(72) Inventors: Omar Ayyub, Germantown, MD (US); Venkata Velvadapu, Germantown, MD (US); James Masciotti, Germantown, MD (US); Philip Huffstetler, Germantown, MD (US)

(73) Assignee: Senseonics, Incorporated, Germantown, MD (US)

(21) Appl. No.: 19/215,473

(22) Filed: May 22, 2025

Related U.S. Application Data
(60) Provisional application No. 63/663,882, filed on Jun. 25, 2024.

(51) Int. Cl. A61B 5/1459; A61B 5/145

(52) U.S. Cl. CPC A61B 5/1459; A61B 5/14532; A61B 2562/12; A61B 2562/16; A61B 2562/185

(57) ABSTRACT

An encasement material that resists degradation, including yellowing. The encasement material may be used, for example, in an apparatus including a housing, circuitry at least partially within the housing, that is implanted into an animal (e.g. a human) to continuously monitor the concentrations of specific analytes, such as glucose. The encasement material allows for the efficient transmission of excitation and emission light to and from an analyte indicator in the device. The encasement material includes additives that resist and inhibit degradation of the encasement material thereby improving the longevity and life of the device. Also, methods for forming the encasement material and incorporating the encasement material within an apparatus.

### CROSS-REFERENCE TO RELATED APPLICATION

The present application claims the benefit of priority to U.S. Provisional Application Ser. No. 63/663,882, filed on Jun. 25, 2024, which is incorporated herein by reference in its entirety.

### BACKGROUND

### Field of Invention

The present invention relates generally to apparatuses utilize an encasement material with one or more additives that inhibit a reduction in transmissivity of the encasement material over time.

### Discussion of the Background

An apparatus may be implanted (partially or fully) within a living animal (e.g., a human). The apparatus may be, for example, a sensor used to measure an analyte (e.g., glucose, oxygen, cardiac markers, low-density lipoprotein (LDL), high-density lipoprotein (HDL), or triglycerides) in a medium (e.g., interstitial fluid (ISF), blood, or intraperitoneal fluid) within the living animal. The sensor may include one or more light sources (e.g., light-emitting diodes (LEDs) or other light emitting elements), analyte indicator molecules, and one or more photodetectors (e.g., one or more photodiodes, one or more phototransistors, one or more photoresistors, and/or one or more other photosensitive elements). Examples of implantable sensors employing indicator molecules to measure an analyte are described in, for example, U.S. Pat. Nos. 5,517,313 and 5,512,246, which are incorporated herein by reference in their entireties.

The indicator molecules in a graft (e.g., a layer, hydrogel, or matrix). For example, if the apparatus is an implantable analyte sensor, indicator molecules (e.g., fluorescent indicator molecules) may reversibly bind an analyte and, when irradiated with excitation light (e.g., light having a wavelength of approximately 378 nm), emit an amount of light (e.g., light in the range of 380 to 600 nm) that depends on whether analyte is bound to the indicator molecule.

An apparatus may include an encasement material that encases circuitry including the one or more light sources and/or the one or more photodetectors. The encasement material may allow the transmission of excitation light from a light source to the analyte indicator molecules and/or emission light from the analyte indicator molecules to the photodetector. Improved encasement materials for apparatuses such as implantable sensor are needed.

### SUMMARY

Overtime, the encasement material may begin to degrade or “yellow”, which can impede the transmission of excitation light and/or emission light through the encasement material and/or cause the formation of fluorescent compounds, both of which can reduce the accuracy of the apparatus and decrease the longevity of the apparatus. Aspects of the invention may improve the accuracy and/or longevity of apparatuses by (1) including in the encasement material one or more additives configured to inhibit (a) a reduction in transmissivity of the encasement material over time and/or (b) formation of fluorescent compounds in the encasement material over time and/or (2) making the encasement material a fully aliphatic system.

One aspect of the invention may provide an apparatus including a housing, circuitry, and an encasement material. The circuitry may be at least partially within the housing. The encasement material may be at least partially within the housing. The encasement material may include one or more additives configured to inhibit a reduction in transmissivity of the encasement material over time.

In some aspects, the one or more additives may be configured to, in inhibiting the reduction of in transmissivity of the encasement material over time, inhibit oxidation of the encasement material. In some aspects, the one or more additives may be configured to, in inhibiting the reduction of in transmissivity of the encasement material over time, inhibit the formation of colored compounds in the transition material. In some aspects, the one or more additives may be further configured to inhibit the formation of fluorescent compounds in the encasement material.

In some aspects, the one or more additives may be one or more antioxidant compounds. In some aspects, the one or more antioxidant compounds may include hindered phenols, hindered amine light stabilizers (HALS), thioethers, phosphites, and/or metal deactivators. In some aspects, the antioxidant compounds may include octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, N′1,N′12-bis(2-hydroxybenzoyl) dodecanedihydrazide, and (ethane-1,2-diylbis(oxy))bis(ethane-2,1-diyl) bis(3-(3-(tert-butyl)-4-hydroxy-5-methylphenyl) propanoate).

In some aspects, the encasement material may include a fully aliphatic system.

Another aspect of the invention may provide an apparatus including a housing, circuitry, and an encasement material. In some aspects, the circuitry may be at least partially within the housing. In some aspects, encasement material may be at least partially within the housing. In some aspects, the encasement material may be composed of a fully aliphatic system.

In some aspects, the fully aliphatic system may include 3,4-epoxycyclohexylmethyl 3,4-epoxycyclohexanecarboxylate and 3-aminomethyl-3,5,5-trimethylcyclohexylamine. In some aspects, the fully aliphatic system may be configured to, in inhibiting the reduction in the transmissivity of the encasement material, inhibit oxidation of the encasement material. In some aspects, the fully aliphatic system may be configured to, in inhibiting the reduction in the transmissivity of the encasement material, inhibit formation of colored compounds in the encasement material. In some aspects, the fully aliphatic system may be further configured to inhibit formation of fluorescent compounds in the encasement material.

In some aspects, the apparatus may further include an analyte indicator. In some aspects, the analyte indicator may cover at least a portion of an exterior surface of the housing. In some aspects, the analyte indicator may be configured to emit an amount of emission light that is indicative of an amount or concentration of an analyte in proximity to the analyte indicator. In some aspects, the circuitry may include a photodetector. In some aspects, the photodetector may be configured to detect the emission light that reaches the photodetector after passing through the encasement material. In some aspects, the encasement material may pass at least 90% of the emission light.

In some aspects, the circuitry may include a light source. In some aspects, the light source may be configured to emit excitation light that reaches the analyte indicator after passing through the encasement material. In some aspects, the analyte indicator may be configured to emit the emission light in response to receiving the excitation light. In some aspects, the encasement material may pass at least 90% of the excitation light.

In some aspects, the circuitry may comprise a power source. In some aspects, the circuitry may comprise an antenna. In some aspects, the antenna may be configured to receive power and/or communicate data.

In some aspects, the encasement material may adhere to the circuitry. In some aspects, the encasement material may adhere to the housing.

In some aspects, the housing can be composed of a polymer of methyl methacrylate (PMMA). In some aspects, the encasement material may be composed of an epoxy.

In some aspects, the encasement materials may have an initial transmissivity at a time of implant of the apparatus. In some aspects, the encasement material may retain at least 90% of the initial transmissivity at 120 days following the time of implant. In some aspects, the encasement material may retain at least 75% of the initial transmissivity at one year following the time of implant.

In some aspects, the fluorescent compounds of the encasement material may produce an initial amount of fluorescence at a time of implant of the apparatus. In some aspects, the encasement material may prevent an increase of fluorescence produced by fluorescent compounds of the encasement material of more than 60% of the initial amount of fluorescence at three months following the time of implant. In some aspects, the encasement material may prevent an increase of the fluorescence produced by the fluorescent compounds of the encasement material of more than about 150% of the initial amount of fluorescence at one year following the time of implant.

In some aspects, the encasement material may encase at least a portion of the circuitry.

Still another aspect of the invention may provide a method of manufacturing an apparatus. The method may include placing circuitry at least partially within a housing of the apparatus and placing an encasement material at least partially within the housing. In some aspects, the encasement material may include one or more additives configured to inhibit a reduction in transmissivity of the encasement material.

In some aspects, the one or more additives may be configured to, in inhibiting the reduction in the transmissivity of the encasement material, inhibit oxidation of the encasement material. In some aspects, the one or more additives may be configured to, in inhibiting the reduction in the transmissivity of the encasement material, inhibit formation of colored compounds in the encasement material. In some aspects, the one or more additives may be further configured to inhibit formation of fluorescent compounds in the encasement material.

In some aspects, the one or more additives may include one or more antioxidant compounds. In some aspects, the one or more antioxidant compound may include hindered phenols, hindered amine light stabilizers (HALS), thioethers, phosphites, and/or metal deactivators. In some aspects, the one or more antioxidant compounds may include octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

In some aspects, the encasement material may include a fully aliphatic system.

Yet another aspect of the invention may provide a method of manufacturing an apparatus. The method may include placing circuitry at least partially within a housing of the apparatus and placing an encasement material at least partially within the housing. In some aspects, the encasement material may be composed of a fully aliphatic system.

In some aspects, the fully aliphatic system may include 3,4-epoxycyclohexylmethyl 3,4-epoxycyclohexanecarboxylate and 3-aminomethyl-3,5,5-trimethylcyclohexylamine. In some aspects, the fully aliphatic system may be configured to, in inhibiting the reduction in the transmissivity of the encasement material, inhibit oxidation of the encasement material. In some aspects, the fully aliphatic system may be configured to, in inhibiting the reduction in the transmissivity of the encasement material, inhibit formation of colored compounds in the encasement material. In some aspects, the fully aliphatic system may be further configured to inhibit formation of fluorescent compounds in the encasement material.

In some aspects, the method may include, before using the encasement material to encase at least the portion of the circuitry, adding metal scavenger molecules to the encasement material and then filtering the encasement material to remove metal scavenger molecules.

In some aspects, the apparatus may include an analyte indicator that covers at least a portion of an exterior surface of the housing. In some aspects, the analyte indicator may be configured to emit an amount of emission light that is indicative of an amount or concentration of an analyte in proximity to the analyte indicator. In some aspects, the circuitry may include a photodetector. In some aspects, the photodetector may be configured to detect the emission light that reaches the photodetector after passing through the encasement material. In some aspects, the circuitry may include a light source configured to emit excitation light that reaches the analyte indicator after passing through the encasement material. In some aspects, the analyte indicator may be configured to emit the emission light in response to receiving the excitation light. In some aspects, the encasement material may pass at least 90% of the excitation light.

In some aspects, the encasement material may have an initial transmissivity at a time of implant of the apparatus. In some aspects, the encasement material may retain at least 90% of the initial transmissivity at 120 days following the time of implant. In some aspects, the encasement material may retain at least 75% of the initial transmissivity at one year following the time of implant.

In some aspects, fluorescent compounds of the encasement material may produce an initial amount of fluorescence at a time of implant of the apparatus. In some aspects, the encasement material may prevent an increase of the fluorescence produced by the fluorescent compounds of the encasement material of more than 60% of the initial amount of fluorescence at three months following the time of implant. In some aspects, the encasement material may prevent an increase of the fluorescence produced by the fluorescent compounds of the encasement material of more than 150% of the initial amount of fluorescence at one year following the time of implant.

In some aspects, placing the encasement material at least partially within the housing may include using the encasement material to encase at least a portion of the circuitry.

Still another aspect of the invention may provide a method that includes using an analyte indicator that covers at least a portion of an exterior surface of a housing of an apparatus to emit an amount of emission light that is indicative of an amount or concentration of an analyte in proximity to the analyte indicator. The method may include using a photodetector to detect the emission light that reaches the photodetector. Circuitry may include the photodetector, and the circuitry may be at least partially within the housing. The encasement material may be at least partially within the housing. The encasement material may include one or more additives configured to inhibit a reduction in transmissivity of the encasement material. The detected emission light may reach the photodetector after passing through the encasement material.

In some aspects, the one or more additives may be configured to, in inhibiting the reduction in the transmissivity of the encasement material, inhibit oxidation of the encasement material. In some aspects, the one or more additives may be configured to, in inhibiting the reduction in the transmissivity of the encasement material, inhibit formation of colored compounds in the encasement material. In some aspects, the one or more additives may be configured to further inhibit formation of fluorescent compounds in the encasement material.

In some aspects, the one or more additives may include one or more antioxidant compounds. In some aspects, the one or more anti-oxidant compound may include hindered phenols, hindered amine light stabilizers (HALS), thioethers, phosphites, and/or metal deactivators. In some aspects, the one or more antioxidant compounds may include octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

In some aspects, the encasement material may include a fully aliphatic system.

Yet another aspect of the invention may provide a method that includes using an analyte indicator that covers at least a portion of an exterior surface of a housing of an apparatus to emit an amount of emission light that is indicative of an amount or concentration of an analyte in proximity to the analyte indicator. The method may include using a photodetector to detect the emission light that reaches the photodetector. Circuitry may include the photodetector, and the circuitry may be at least partially within the housing. The encasement material may be at least partially within the housing. The encasement material may be composed of a fully aliphatic system. In some aspect the detected emission light reaches the photodetector after passing through the encasement material.

In some aspects, the fully aliphatic system may include 3,4-epoxycyclohexylmethyl 3,4-epoxycyclohexanecarboxylate and 3-aminomethyl-3,5,5-trimethylcyclohexylamine. In some aspects, the fully aliphatic system may be configured to inhibit oxidation of the encasement material. In some aspects, the fully aliphatic system may be configured to inhibit formation of colored compounds in the encasement material. In some aspects, the fully aliphatic system may be configured to inhibit formation of fluorescent compounds in the encasement material.

In some aspects, the circuitry includes a light source. In some aspects, the light source emits excitation light that reaches the analyte indicator after passing through the encasement material. In some aspects, the analyte indicator may be configured to emit the emission light in response to receiving the excitation light. In some aspects, the encasement material passes at least 90% of the excitation light.

In some aspects, the circuitry may include a power source. In some aspects, the circuitry may include an antenna. In some aspects, the antenna can be configured to receive power and/or communicate data.

In some aspects, the encasement material may adhere to the circuitry. In some aspects, the encasement material may adhere to the housing.

In some aspects, the housing may include a polymer of methyl methacrylate (PMMA). In some aspects, the encasement material may include an epoxy.

In some aspects, the encasement material may have an initial transmissivity at a time of implant of the apparatus, and the encasement material may retain at least 90% of the initial transmissivity at 120 days following the time of implant. In some aspects, the encasement material may have an initial transmissivity a time of at implant of the apparatus, and the encasement material may retain at least 75% of the initial transmissivity at one year following the time of implant. In some aspects, fluorescent compounds of the encasement material may produce an initial amount of fluorescence at a time of implant of the apparatus, and the encasement material may prevent an increase of the fluorescence produced by the fluorescent compounds of the encasement material of more than 60% of the initial amount of fluorescence at three months following the time of implant. In some aspects, fluorescent compounds of the encasement material may produce an initial amount of fluorescence at a time of implant of the apparatus, and the encasement material may prevent an increase of the fluorescence produced by the fluorescent compounds of the encasement material of more than 150% of the initial amount of fluorescence at one year following to the time of implant.

In some aspects, the encasement material may encase at least a portion of the circuitry.

Another aspect of the invention may provide a method to manufacture an apparatus that may include forming an encasement material mixture, adding one or more metal scavenging molecules to the encasement material mixture, filtering the mixture to remove at least a portion of the metal scavenging molecules, allowing the encasement material mixture to harden.

In some aspects, the encasement material mixture may include one or more additives configured to inhibit a reduction in transmissivity of the encasement material. In some aspects, the one or more additives may be configured inhibit oxidation of the encasement material. In some aspects, the one or more additives may be configured to inhibit formation of colored compounds in the encasement material. In some aspects, the one or more additives may be configured to inhibit formation of fluorescent compounds in the encasement material.

In some aspects, the one or more additives may include one or more antioxidant compounds. In some aspects, the one or more anti-oxidant compound may include hindered phenols, hindered amine light stabilizers (HALS), thioethers, phosphites, and/or metal deactivators. In some aspects, the one or more antioxidant compounds may include octadecyl 3-(3,5-di-tert-butyl-4-hydroxyphenyl) propionate, pentaerythritol tetrakis(3,5-di-tert-butyl-4-hydroxyhydrocinnamate′, 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, an′/or′N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

In some aspects, the encasement material may include a fully aliphatic system. In some aspects, the fully aliphatic system may include 3,4-epoxycyclohexylmethyl 3,4-epoxycyclohexanecarboxylate and 3-aminomethyl-3,5,5-trimethylcyclohexylamine. In some aspects, the fully aliphatic system may be configuring to inhibit oxidation of the encasement material. In some aspects, the fully aliphatic system may be configured to inhibit formation of colored compounds in the encasement material. In some aspects, the fully aliphatic system may be configured to inhibit formation of fluorescent compounds in the encasement material.

In some aspects, the encasement material may pass at least 90% of the excitation light. In some aspects, the housing may include a polymer of methyl methacrylate (PMMA). In some aspects, the encasement material may include an epoxy.

In some aspects, the encasement material may have an initial transmissivity at implant. In some aspects, the encasement material may have an initial transmissivity at a time of implant of the apparatus, and the encasement material retains at least 90% of the initial transmissivity at 120 days following the time of implant. In some aspects, the encasement material may have an initial transmissivity at a time of implant of the apparatus, and the encasement material may retain at least 75% of the initial transmissivity at one year following the time of implant. In some aspects, fluorescent compounds of the encasement material may produce an initial amount of fluorescence at a time of implant of the apparatus, and the encasement material may prevent an increase of the fluorescence produced by the fluorescent compounds of the encasement material of more than 60% of the initial amount of fluorescence at three months following the time of implant. In some aspects, fluorescent compounds of the encasement material may produce an initial amount of fluorescence at a time of implant of the apparatus, and the encasement material may prevent an increase of the fluorescence produced by the fluorescent compounds of the encasement material of more than 150% of the initial amount of fluorescence at one year following the time of implant.

Another aspect of the invention may provide a method to manufacture an apparatus. The method may include adding one or more additives to an encasement material formulation and curing the encasement material formulation to form the encasement material.

In some aspects, the one or more additives may be configured to inhibit oxidation of the encasement material. In some aspects, the one or more additives may be configured to slow formation of colored compounds in the encasement material. In some aspects, the one or more additives may be configured to inhibit formation of fluorescent compounds in the encasement material.

In some aspects, the one or more additives may include one or more antioxidant compounds. In some aspects, one or more antioxidant compound may include hindered phenols, hindered amine light stabilizers (HALS), thioethers, phosphites, and/or metal deactivators. In some aspects, the one or more antioxidant compounds may include octadecyl 3-(3,5-di-tert-butyl-4-hydroxyphenyl) propionate, pentaerythritol tetrakis(3,5-di-tert-butyl-4-hydroxyhydroci′namate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzam′de,′and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

In some aspects, the encasement material may further include a fully aliphatic system. In some aspects, the fully aliphatic system may include 3,4-epoxycyclohexylmethyl 3,4-epoxycyclohexanecarboxylate and 3-aminomethyl-3,5,5-trimethylcyclohexylamine. In some aspects, the fully aliphatic system may be configured to inhibit oxidation of the encasement material. In some aspects, the fully aliphatic system may be configured to inhibit formation of colored compounds in the encasement material. In some aspects, the fully aliphatic system may be configured to inhibit formation of fluorescent compounds in the encasement material.

In some aspects, the encasement material may have an initial transmissivity at implant. In some aspects, the encasement material may have an initial transmissivity at a time of implant of the apparatus, and the encasement material retains at least 90% of the initial transmissivity at 120 days following the time of implant. In some aspects, the encasement material may have an initial transmissivity at a time of implant of the apparatus, and the encasement material retains at least 75% of the initial transmissivity at one year following the time of implant. In some aspects, fluorescent compounds of the encasement material may produce an initial amount of fluorescence at a time of implant of the apparatus, and the encasement material may prevent an increase of the fluorescence produced by the fluorescent compounds of the encasement material of more than 60% of the initial amount of fluorescence at three months following the time of implant. In some aspects, fluorescent compounds of the encasement material may produce an initial amount of fluorescence at a time of implant of the apparatus, and the encasement material may prevent an increase of the fluorescence produced by the fluorescent compounds of the encasement material of more than 150% of the initial amount of fluorescence at one year following the time of implant.

### BRIEF DESCRIPTION OF THE DRAWINGS

The accompanying drawings, which are incorporated herein and form part of the specification, illustrate various, non-limiting aspects of the present invention. In the drawings, like reference numbers indicate identical or functionally similar elements.

FIG. 1 is a schematic view illustrating a system embodying aspects of the present invention.

FIG. 2A is an exploded view of an apparatus of the system according to some aspects of the present invention.

FIG. 2B shows a power source and a coupler of the apparatus according to some aspects of the present invention.

FIG. 2C shows circuitry at least partially within a housing of the apparatus according to some aspects of the present invention.

FIGS. 2D and 2E show the housing in contact with the coupler of the apparatus according to some aspects of the present invention.

FIG. 3 is a schematic view illustrating a system embodying aspects of the present invention.

FIG. 4 is a chart comparing the change in transmission efficiencies of the encasement material over time with and without one or more additives embodying aspects of the present invention.

FIG. 5 is a chart comparing the change in fluoresce of the encasement material over time with and without one or more additives embodying aspects of the present invention.

FIG. 6 is a flow chart illustrating a process according to some aspects.

FIG. 7 is a flow chart illustrating a process according to some aspects.

### DETAILED DESCRIPTION

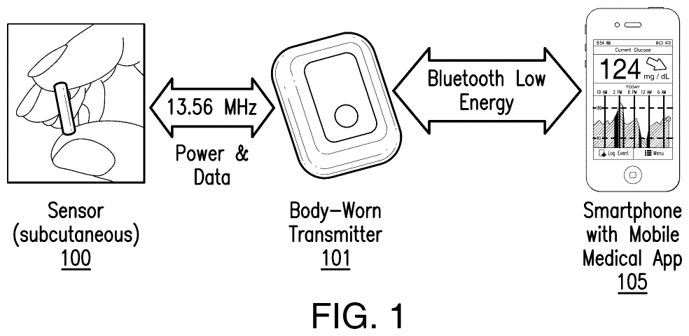

FIG. 1 is a schematic view of an exemplary system 50 embodying aspects of the present invention. In some aspects, the system 50 may be an analyte monitoring system. In some aspects, the system 50 may be a continuous analyte monitoring system (e.g., a continuous glucose monitoring system). In some aspects, the system 50 may include one or more of an apparatus 100, an external device 101, and a display device 107.

In some aspects, the apparatus 100 may be an implantable device. In some aspects, the apparatus 100 may be a wireless implantable device. In some aspects, the apparatus 100 may be a sensor (e.g., an analyte sensor). In some aspects, the apparatus 100 may include one or more optical sensors (e.g., one or more fluorometers). In some aspects, the apparatus 100 may be chemical or biochemical sensors. In some aspects, the apparatus 100 may be a radio frequency identification (RFID) device. In some aspects, the apparatus 100 may be a small, fully subcutaneously implantable sensor that detects the presence, amount, and/or concentration of an analyte (e.g., glucose, oxygen, cardiac markers, low-density lipoprotein (LDL), high-density lipoprotein (HDL), or triglycerides) in a medium (e.g., interstitial fluid) of a living animal (e.g., a living human). However, this is not required, and, in some alternative aspects, the apparatus 100 may be a partially implantable (e.g., transcutaneous) device or a fully external sensor. In addition, although aspects of the invention are described with respect to an analyte monitoring system in which the apparatus 100 is an analyte sensor, this is not required. In some alternative aspects, the apparatus 100 is not a sensor and is instead a different type of apparatus, such as, for example and without limitation, an insulin pump (e.g., an implantable insulin pump), a pacemaker (e.g., an implantable pacemaker), or electrical/heat therapy device (e.g., an implantable electrical/heat therapy device).

In some aspects, the external device 101 may be an externally worn device (e.g., attached via an armband, wristband, waistband, or adhesive patch). In some aspects, the external device 101 may remotely communicate with the apparatus 100 (e.g., via near field communication (NFC)). In some aspects, the external device 101 may communicate with the apparatus 100 to initiate and/or read data (e.g., measurements) from the apparatus 100. In some aspects, the external device 101 may be a transceiver. In some aspects, the external device 101 may be a smartphone (e.g., an NFC-enabled smartphone). In some aspects, the external device 101 may communicate information (e.g., one or more analyte measurements) wirelessly (e.g., via a Bluetooth™ communication standard such as, for example and without limitation Bluetooth Low Energy) to an application running on a display device 107 (e.g., smartphone). In some aspects, the display device 107 may additionally or alternatively communicate directly with the apparatus 100 (e.g., via near field communication (NFC)). In some aspects, the display device 107 may communicate with the apparatus 100 to initiate and/or read data (e.g., measurements) from the apparatus 100.

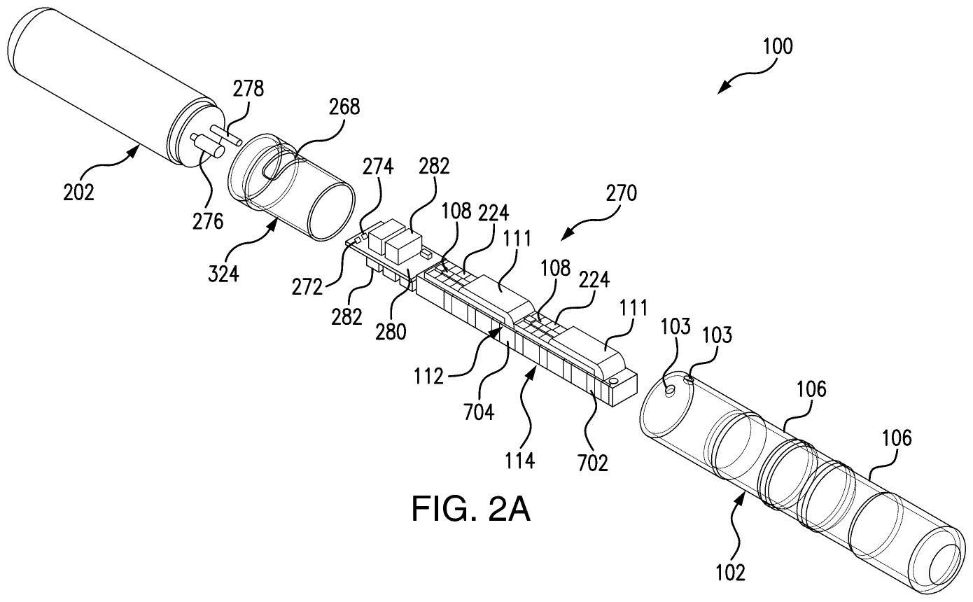

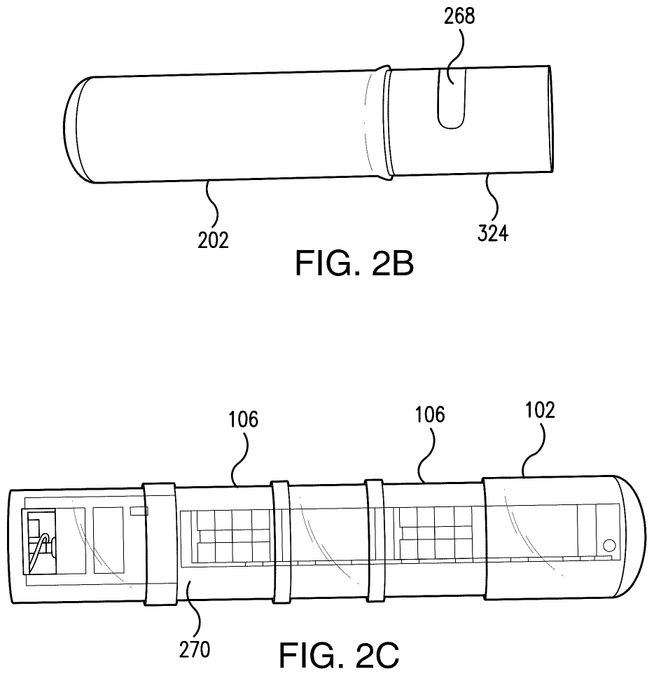

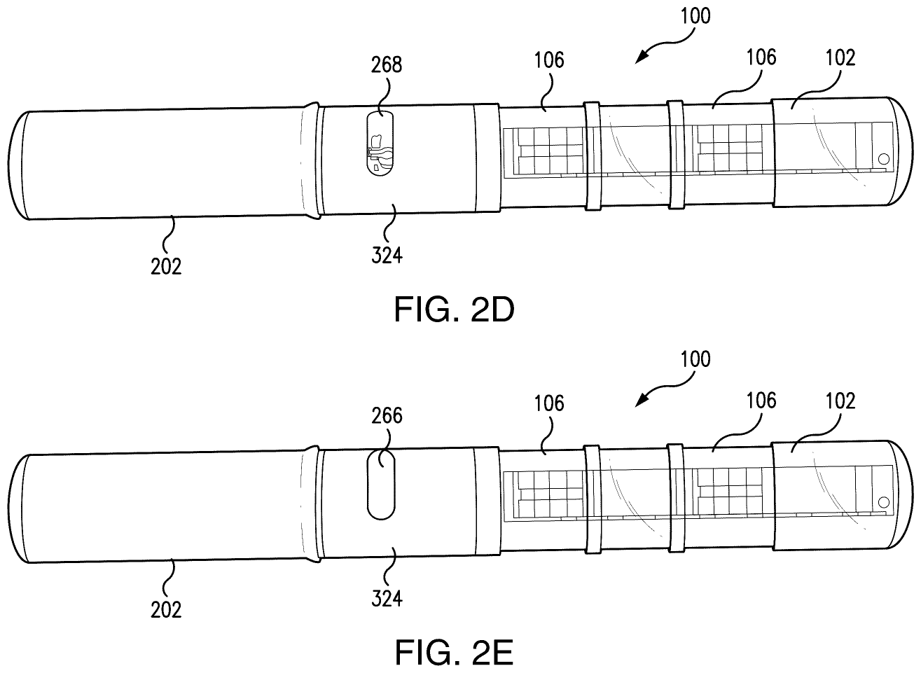

FIG. 2A is an exploded view of the apparatus 100 of the system 50 according to some aspects. In some aspects, as shown in FIG. 2A, the apparatus 100 may include a housing 102, circuitry 270, a power source 202, first and second electrically conductive leads 276 and 278, and/or a coupler 324. In some aspects, as shown in FIG. 2B, a first end of the coupler 324 may be attached to the power source 202. In some aspects, as shown in FIG. 2C, the circuitry 270 may be at least partially within the housing 102. In some aspects, as shown in FIGS. 2D and 2E, at least a portion of the housing 102 may be in contact with a second end of the coupler 324. In some aspects, as shown in FIGS. 2D and 2E, at least a portion of the housing 102 may extend into a second end of the coupler 324.

In some aspects, the housing 102 may be a body, shell, capsule, sleeve, or tube. In some aspects, the housing 102 may be rigid and/or biocompatible. In some aspects, the housing 102 may include a polymer (e.g., PMMA or silicone). However, this is not required, and, in other aspects, different materials and/or shapes may be used for the housing 102.

In some aspects, the apparatus 100 may include one or more analyte indicators 104, which may be, for example, polymer grafts or hydrogels coated, diffused, adhered, embedded, or grown on or in one or more portions of the exterior surface of the housing 102. In some aspects, as shown in FIGS. 2A, 2C, 2D, and 2E, the housing 102 may include one or more cutouts or recesses, and one or more analyte indicators 104 may be located (partially or entirely) in the cutouts or recesses. In some aspects, the one or more analyte indicators 104 may be porous and may allow an analyte (e.g., glucose) in a medium (e.g., interstitial fluid) to diffuse into the one or more analyte indicators 104.

In some aspects, the one or more analyte indicators 104 may have one or more detectable properties (e.g., optical properties) that vary in accordance with the amount or concentration of the analyte in proximity to the indicator element 104. In some aspects, the one or more analyte indicators 104 may include one or more analyte indicator molecules (e.g., fluorescent analyte indicator molecules), which may be distributed throughout the one or more analyte indicators 104. In some aspects, the one or more analyte indicators 104 may be phenylboronic-based analyte indicators. However, a phenylboronic-based analyte indicator is not required, and, in some alternative aspects, the one or more analyte indicators 104 may be different analyte indicators, such as, for example and without limitation, glucose oxidase-based indicators, glucose dehydrogenase-based indicators, and glucose binding protein-based indicators.

In some aspects, the circuitry 270 may include measurement electronics (e.g., optical measurement electronics), one or more circuit components 111 (e.g., analog and/or digital circuit components), an antenna 114, one or more capacitors 282, and/or first and second contact pads 272 and 274. In some aspects, the measurement electronics of the circuitry 270 may include one or more light sources 108 (e.g., one or more light emitting diodes (LEDs)) and one or more photodetectors 224 (e.g., one or more photodiodes, phototransistors, photoresistors, or other photosensitive elements). In some aspects, the one or more light sources 108 may be configured to emit excitation light (e.g., ultraviolet (UV) light) that reaches the one or more analyte indicators 104. In some aspects, the one or more photodetectors 224 may be configured to detect emission light (e.g., fluorescent light) that reaches the one or more photodetectors 224 after being emitted by the one or more analyte indicators 104. In some aspects, the amount of emission light emitted by the one or more analyte indicators 104 may correspond to the amount of analyte (e.g., glucose) in the medium (e.g., interstitial fluid) in proximity to the one or more analyte indicators 104. For example, in some aspects, the analyte may bind reversibly to analyte indicator molecules of the one or more analyte indicators 104, analyte indicator molecules to which the analyte is bound may emit emission light when irradiated by the excitation light, and analyte indicator molecules to which the analyte is not bound may not emit light (or emit only a small amount of light) when irradiated by the excitation light.

In some aspects, as shown in FIG. 2A, the apparatus 100 may include one or more substrates 112. In some aspects, the one or more substrates 112 may be circuit boards (e.g., one or more flexible and/or rigid printed circuit boards (PCBs)). In some aspects, one or more of the circuit components 111 may be mounted or otherwise attached to the one or more substrates 112. However, in some alternative aspects, the one or more substrates 112 may be semiconductor substrates having one or more of the circuit components 111 fabricated therein. For instance, the fabricated circuit components may include analog and/or digital circuitry. Also, in some aspects in which the substrate 112 is a semiconductor substrate, in addition to the one or more circuit components fabricated in the semiconductor substrate, one or more circuit components may be mounted or otherwise attached to the semiconductor substrate. In other words, in some semiconductor substrate aspects, a portion or all of the circuit components 111, which may include discrete circuit elements, an integrated circuit (e.g., an application specific integrated circuit (ASIC)) and/or other electronic components (e.g., a non-volatile memory), may be fabricated in the semiconductor substrate with the remainder of the circuit components 111 secured to the semiconductor substrate, which may provide communication paths between the various secured components.

In some aspects, as shown in FIG. 2A, the measurement electronics of the circuitry 270 may be mounted on and/or fabricated in the one or more substrates 112. In some aspects, as shown in FIG. 2A, the one or more substrates 112 may include (i) a first set of one or more light sources 108 and one or more photodetectors 224 and (ii) a second set of one or more light sources 108 and one or more photodetectors 224. In some aspects, the one or more light sources 108 may be mounted on the one or more substrates 112, the one or more photodetectors 224 may be fabricated in the substrate 112, and all or a portion of the circuit components 111 may be fabricated within the substrate 112.

In some aspects, as shown in FIG. 2A, the antenna 114 may be an inductor including a conductor 702 in the form of a coil and a magnetic core 704. In some aspects, the core 704 may be, for example and without limitation, a ferrite core. In some aspects, the antenna 114 may be, for example, a ferrite-based micro-antenna. In some aspects, as illustrated in FIG. 2A, the one or more substrates 112 of the apparatus 100 may be attached to the antenna 114. In some aspects, the circuit components 111 of the substrates 112 may be connected electrically to the antenna 114. In some aspects, the apparatus 100 may use the antenna 114 to communicate data (e.g., measurement data) to the external device 101 and/or the display device 107. In some aspects, the apparatus 100 may use the antenna 114 for NFC.

In some aspects, as shown in FIG. 2A, the apparatus 100 may include a PCB 280. In some aspects, the one or more capacitors 282 of the circuitry 270 may be mounted on the PCB 280. In some aspects, the PCB 280 may include the first and second contact pads 272 and 274 of the circuitry 270. In some aspects, the circuit components 111 of the substrates 112 and/or the antenna 114 may be connected electrically to the one or more capacitors 282 and/or the first and second contact pads 272 and 274.

In some aspects, the apparatus 100 (e.g., the circuitry 270 of the apparatus 100) may be powered at least partially by the power source 202. In some aspects, the power source 202 may be a charge storage device (e.g., a battery, capacitor, or super capacitor). In some aspects, at least the exterior of the power source 202 may be made of a biocompatible material such as, for example and without limitation, stainless steel or a titanium alloy. In some aspects, the power source 202 may be a titanium-cased, hermetically-sealed battery. In some aspects, as shown in FIGS. 2A, 2D, and 2E, the circuitry 270 of the apparatus 100 may extend away from the power source 202 along the longitudinal axis of the power source 202.

In some aspects, the power source 202 may include first and second terminals (e.g., a positive terminal (cathode) and a negative terminal (anode)). In some aspects, the first and second electrically conductive leads 276 and 278 may be connected electrically to the first and second terminals, respectively, of the power source 202. In some aspects, the electrically conductive leads 276 and 278 may electrically connect the first and second terminals, respectively, of the power source 202 to the circuitry 270 of the apparatus 100. In some aspects, the electrically conductive leads 276 and 278 may be rods or beams including or made out of a conductive material.

In some alternative aspects the apparatus 100 may not have an internal power source and/or may powered completely or partially through induction by an external power source (e.g., the external device 101).

In some aspects, as shown in FIGS. 2A, 2B, 2D, and 2E, the coupler 324 may be a flange. In some aspects, as shown in FIGS. 2B, 2D, and 2E, the coupler 324 may be attached to the power source 202. In some aspects, the coupler 324 may be welded (e.g., laser welded) to the power source 202. In some aspects, the coupler 324 may enclose the first and second terminals of the power source 202. In some aspects, as shown in FIGS. 2D and 2E, the coupler 324 may be between the housing 102 and the power source 202. In some aspects, as shown in FIG. 2E, the apparatus 100 may further include a cap 266 over the one or more of the couplers 324.

In some aspects, the coupler 324 may have a generally cylindrical shape. However, other shapes (e.g., a generally rectangular prism shape) may be used in alternative aspects. In some aspects, the coupler 324 may be made of a biocompatible material such as, for example and without limitation, glass, ceramic, stainless steel, titanium, or a titanium alloy. In some aspects, the coupler 324 may include a flat surface that abuts and is attached to the power source 202.

In some aspects, as shown in FIGS. 2A, 2B, 2D, and 2E, the coupler 324 may include one or more openings 268 through which the first and second electrically conductive leads 276 and 278 are capable of being laser welded to the first and second contact pads 272 and 274, respectively, of the circuitry 270. In some aspects, the housing 102 may include one or more openings 103 through which the first and second electrically conductive leads 276 and 278 are capable of being laser welded to the first and second contact pads 272 and 274, respectively, of the circuitry 270.

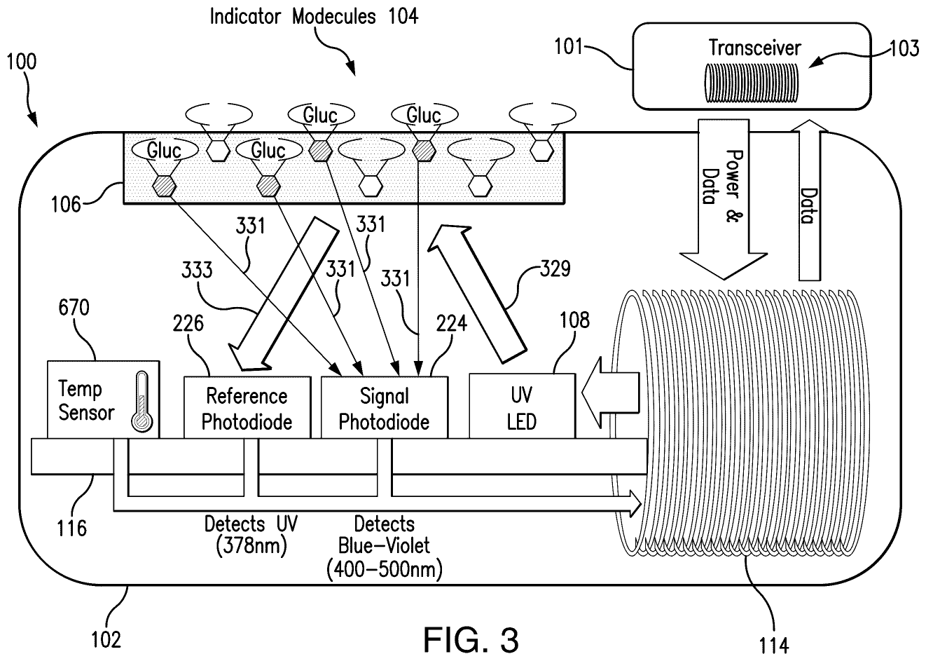

In some aspects, as shown in FIGS. 2C-3, the apparatus 100 may further include an encasement material 109 that encases at least a first portion of the circuitry 270 in the housing 102. In some aspects, the first portion of the circuitry 270 may include the one or more light sources 108 and the one or more photodetectors 224. In some aspects, the encasement material 109 may include an epoxy (e.g., a water-resistant epoxy).

In some aspects, the encasement material 109 may not encase the first and second contact pads 272 and 274. In some aspects, the apparatus 100 may further include an additional encasement material that encases the first and second electrically conductive leads 276 and 278 and a second portion of the circuitry 270. In some aspects, the second portion of the circuitry 270 may include the first and second contact pads 272 and 274. In some aspects, the encasement material 109 and any additional encasement material may be different. In some alternative aspects, the encasement material 109 and any additional encasement materials may be the same. In some aspects, the additional encasement material may include an epoxy (e.g., a water-resistant epoxy). In some aspects, the encasement material 109 may fill a first portion of the housing 102, and the additional encasement material may fill the coupler 324 and a second portion of the housing 102 that is not filled by the encasement material 109.

In some alternative aspects, instead of encasement material 109 and any additional encasement materials, the encasement material 109 may encase the circuitry 270 and the first and second electrically conductive leads 276 and 278 and may fill the housing 102 and the coupler 324.

In some aspects, the encasement material 109 may be physically adhered to one or more components of the apparatus 100. In some aspects, the encasement material 109 may adhere to the circuitry 270. In some aspects, the encasement material 109 may adhere to the housing 102.

In some aspects, the housing 102 may be composed of one or more materials. In some aspects the housing 102 may include a polymer. In some aspects, the housing 102 may include a polymer of methyl methacrylate (PMMA).

In some aspects, the encasement material 109 may include one or more materials. In some aspects, the encasement material 109 may include one or more polymers. In some aspects, the encasement material 109 may include an epoxy. In some aspects, the encasement material 109 may include Epo Tek 301-2 epoxide.

In some aspects, the encasement material 109 may include one or more additives. In some aspects, the one or more additives may inhibit reduction in transmissivity of the encasement material 109 overtime (e.g., compared to an encasement material 109 without said additives). In some aspects, the one or more additives may additionally or alternatively be configured to inhibit oxidation of the encasement material 109. In some aspects, the one or more additives may additionally or alternatively be configured to inhibit the formation of colored compounds in the encasement material 109. In some aspects, the one or more additives may additionally or alternatively be configured to inhibit the formation of fluorescent compounds in the encasement material 109. In some aspects, the one or more additives may be configured to inhibit oxidation of the encasement material 109, inhibit the formation of colored materials in the encasement material 109, and inhibit the formation of fluorescent compounds in the encasement material 109. In some aspects, the encasement material 109 may include a mixtures of additives that separately perform one or more of the protective functions above. In some alternative aspects, the encasement material 109 can include a single additive that can perform one or more of the protective functions above.

In some aspects, the one or more additives may include one or more antioxidant compounds. In some aspects, the antioxidant compounds may include hindered phenols, hindered amine light stabilizers (HALS), thioethers, phosphites, and/or metal deactivators, including salts and mixtures thereof. In some aspects, the antioxidant compounds may additionally or alternatively include octadecyl 3-(3,5-di-tert-butyl-4-hydroxyphenyl) propionate, pentaerythritol tetrakis(3,5-di-tert-butyl-4-hydroxyhydroci′namate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl′ben′amide, N′1,N′12-bis(2-hydroxybenzoyl) dodecanedihydrazide, and (ethane-1,2-diylbis(oxy))bis(ethane-2,1-diyl) bis(3-(3-(tert-butyl)-4-hydroxy-5-methylphenyl) propanoate), including salts and mixtures thereof.

In some aspects, the encasement material 109 may be include a fully aliphatic system. In some aspects, the fully aliphatic system may include 3,4-epoxycyclohexylmethyl 3,4-epoxycyclohexanecarboxylate and/or 3-aminomethyl-3,5,5-trimethylcyclohexylamine. In some aspects, the fully aliphatic system may be configured to inhibit oxidation of the encasement material 109. In some aspects, the fully aliphatic system may additionally or alternatively be configured to inhibit formation of colored compounds in the encasement material 109. In some aspects, the fully aliphatic system may additionally or alternatively be configured to inhibit formation of fluorescent compounds in the encasement material 109.

FIG. 3 is a schematic view of the apparatus 100 and external device 101 of the system 50 according to some aspects of the present invention.

In some aspects, the external device 101 may be an electronic device that communicates with the apparatus 100 to power the apparatus 100, provide commands, and/or data to the apparatus 100, and/or receives data from the apparatus 100. In some aspects, the received data may include one or more sensor measurements. In some aspects, the sensor measurements may include, for example and without limitation, one or more light measurements from one or more photodetectors of the apparatus 100 and/or one or more temperature measurements from one or more temperature sensors of the apparatus 100. In some aspects, the external device 101 may calculate analyte (e.g., glucose) concentrations from the measurement information received from the apparatus 100.

In some aspects, as shown in FIG. 3, the external device 101 may include an antenna 114, such as, for example, a coil. In some aspects, the external device 101 may generate an electromagnetic wave or electrodynamic field (e.g., by using a coil) to induce a current in an antenna 114 of the apparatus 100. In some aspects, the apparatus 100 may use the current induced in the antenna 114 to power the apparatus 100. However, this is not required, and, in some alternative aspects, the apparatus 100 may be partially or completely powered by an internal power source (e.g., a battery).

In some aspects, the external device 101 may convey data (e.g., commands) to the apparatus 100. For example, in some aspects, the external device 101 may convey data by modulating the electromagnetic wave generated by the antenna 114 (e.g., by modulating the current flowing through the antenna 114 of the external device 101). In some aspects, the apparatus 100 may detect/extract the modulation in the electromagnetic wave generated by the external device 101. Moreover, the external device 101 may receive data (e.g., one or more sensor measurements) from the apparatus 100. For example, in some aspects, the external device 101 may receive data by detecting modulations in the electromagnetic wave generated by the apparatus 100, e.g., by detecting modulations in the current flowing through the antenna 114 of the external device 101.

In some aspects, as shown in FIG. 3, the analyte indicator 106 (e.g., polymer graft) of the apparatus 100 may include indicator molecules 104. The indicator molecules 104 may be distributed throughout the entire analyte indicator 106 or only throughout one or more portions of the analyte indicator 106. In some aspects, the indicator molecules 104 may include a boronate group. In some aspects, the indicator molecules 104 may be fluorescent indicator molecules (e.g., TFM having the chemical name 9-[N-[6-(4,4,5,5,-tetramethyl-1,3,2-dioxaborolano)-3-(trifluoromethyl)benzyl]-N-[3-(methacrylamido) propylamino]methyl]-10-[N-[6-(4,4,5,5,-tetramethyl-1,3,2-dioxaborolano)-3-(trifluoromethyl)benzyl]-N-[2-(carboxyethyl)amino]methyl]anthracene sodium salt) or light absorbing, non-fluorescent indicator molecules. In some aspects, the indicator molecules 104 may reversibly bind an analyte (e.g., glucose, oxygen, cardiac markers, low-density lipoprotein (LDL), high-density lipoprotein (HDL), or triglycerides). When an indicator molecule 104 has bound an analyte, the indicator molecule may become fluorescent, in which case the indicator molecule 104 is capable of absorbing (or being excited by) excitation light 329 and emitting light 331. In some aspects, the excitation light 329 may have a wavelength of approximately 378 nm, and the emission light 331 may have a wavelength in the range of 380 to 600 nm. When no analyte is bound, the indicator molecule 104 may be only weakly fluorescent.

In some aspects, the apparatus 100 may include a light source 108, which may be, for example, a light emitting diode (LED) or other light source that emits radiation, including radiation over a range of wavelengths that interact with the indicator molecules 104. In other words, the light source 108 may emit the excitation light 329 that is absorbed by the indicator molecules in the matrix layer/polymer 104. As noted above, in some aspects, the light source 108 may emit excitation light 329 at a wavelength of approximately 378 nm.

In some aspects, the apparatus 100 may also include one or more photodetectors (e.g., photodiodes, phototransistors, photoresistors or other photosensitive elements). For example, as illustrated in FIG. 3, apparatus 100 may have a first photodetector 224 and a second photodetector 226. However, this is not required, and, in some alternative aspects, the apparatus 100 may only include the first photodetector 224. In the case of a fluorescence-based sensor, the one or more photodetectors may be sensitive to fluorescent light emitted by the indicator molecules 104 such that a signal is generated by a photodetector (e.g., photodetector 224) in response thereto that is indicative of the level of fluorescence of the indicator molecules and, thus, the amount of analyte of interest (e.g., glucose).

In some aspects, some part of the excitation light 329 emitted by the light source 108 may be reflected from the analyte indicator 106 back into the apparatus 100 as reflection light 333, and some part of the absorbed excitation light may be emitted as emitted (fluoresced) light 331. In some aspects, the emitted light 331 may have a different wavelength than the wavelength of the excitation light 329. The reflected light 333 and emitted (fluoresced) light 331 may be absorbed by the one or more photodetectors (e.g., first and second photodetectors 224 and 226) within the body of the apparatus 100.

In some aspects, each of the one or more photodetectors may be covered by a filter 112 that allows only a certain subset of wavelengths of light to pass through. In some aspects, the one or more filters 112 may be thin glass filters. In some aspects, the one or more filters 112 may be thin film (e.g., dichroic) filters deposited on the glass and may pass only a narrow band of wavelengths and otherwise reflect most of the received light. In some aspects, the filters may be thin film (dichroic) filters deposited directly onto the photo detectors and may pass only a narrow band of wavelengths and otherwise reflect most of the light received thereby. The filters 112 may be identical (e.g., both filters 112 may allow signals to pass) or different (e.g., one filter 112 may be a reference filter and another filter 112 may be a signal filter).

In some aspects, the second (reference) photodetector 226 may be covered by a reference photodiode filter that passes light at the same wavelength as is emitted from the light source 108 (e.g., 378 nm). The first (signal) photodetector 224 may detect the amount of fluoresced light 331 that is emitted from the molecules 104 in the analyte indicator 106. In some aspects, the peak emission of the indicator molecules 104 may occur around 435 nm, and the first photodetector 224 may be covered by a signal filter that passes light in the range of about 380 nm to 600 nm. In some aspects, higher glucose levels/concentrations may correspond to a greater amount of fluorescence of the molecules 104 in the analyte indicator 106, and, therefore, a greater number of photons striking the first photodetector 224.

In some aspects, the encasement material 109 may partially or completely encase the circuitry 270 in the housing 102. In some aspects, the encasement material 109 may allow passage of excitation light 329 from the light source 108 to the analyte indicator 106, the passage of emission light 331 from the analyte indicator 106 to the signal photodetector 224, and the passage of reflected light 333 from the analyte indicator 106 to the reference photodetector 226.

In some aspects, the excitation light 329 emitted by the one or more light sources 108 of the circuitry 270 may reach the one or more analyte indicators 104 after passing through the encasement material 109. In some aspects, the emission light 331 emitted by the one or more analyte indicators 106 may reach the one or more photodetectors 224 after passing through the encasement material 109.

In some aspects, the encasement material 109 may allow transmission of some or all of the excitation light. In some aspects, the encasement material 109 may allow a minimum percentage of excitation light 329 to pass. In some aspects, the encasement material 109 may allow the transmission of at least about 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or 100% of the excitation light 109. In some aspects, the encasement material 109 may be optically clear. In some aspects, the encasement material 109 may allow transmission of at least about 90% of the excitation light 329. In some aspects, the encasement material 109 may allow different efficiencies (percentages of original produced light) of transmissions of different wavelengths of excitation light 329. In some aspects, the encasement material 109 may allow transmission of different wavelengths of excitation light at the same efficiency (percentage of original produced light).

In some aspects, the encasement material 109 may allows transmission of some or all of the emission light 331. In some aspects the encasement material 109 may allow a minimum percentage emission light 331 to pass. In some aspects, the encasement material 109 may allow the transmission at least about 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or 100% of the emission light 331. In some aspects, the encasement material 109 may be optically clear. In some aspects, the encasement material 109 may allow transmission of at least about 90% of the emission light 331. In some aspects the encasement material 109 may allow different efficiencies (percentages of original produced light) of transmissions of different wavelengths of emission light 311. In some aspects, the encasement material 109 may allow transmission of different wavelengths of emission light 331 at the same efficiency (percentage of original produced light).

In some aspects, the encasement material 109 may allow the efficient transmission of certain wavelengths of excitation light 329. In some aspects, excitation light wavelengths 329 contemplated by the current invention may be between about 250 nm and about 900 nm. Other ranges of excitation light wavelengths 329 contemplated by the invention may be between about 300 nm and about 800 nm, between about 350 nm and about 750 nm, between about 360 nm and about 700 nm, between about 370 and about 650 nm. In some aspects, the ranges of excitation light wavelengths 329 may be between 380 nm and 600 nm.

In some aspects, the encasement material 109 may allow the efficient transmission of certain wavelengths of emission light 331. In some aspects, emission light wavelengths 331 contemplated by the current invention may be between about 250 nm and about 900 nm. Other ranges of emission light wavelengths 331 contemplated by the invention may be between about 300 nm and about 800 nm, between about 350 nm and about 750 nm, between about 360 nm and about 700 nm, between about 370 and about 650 nm. In an aspect of the invention, the ranges of emission light wavelengths 331 may be between 380 nm and 600 nm.

In some aspects, the encasement material 109 may have an initial transmissivity (e.g., efficiency of the transmission of excitation and emission light) at the time of implant. In some aspects, the encasement material 109 may resist a decrease of transmissivity over time. In some aspects, the encasement material may retain at least 10%, 20%, 30%, 40%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% transmissivity at 120 days following implantation compared to the initial transmissivity. In an aspect, the encasement material 109 may retain at least 90% of the initial transmissivity at 120 days following implant compared to the initial transmissivity. In some aspect, the encasement material 109 may retain at least 10%, 20%, 30%, 40%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% transmissivity at one year following implantation. In an aspect, the encasement material 109 may retain at least 75% of the initial transmissivity at one year following implant compared to the initial transmissivity.

In some aspects, the analyte indicator 104 may have an initial fluorescence at the time of implant. In some aspect, the encasement material 109 may inhibit an increase of fluorescence compared to the initial fluorescence at the time of implant. In some aspect, the encasement material 109 may prevent an increase of fluorescence of more than about 10%, 20%, 30%, 40%, 50%, 55%, 60%, 65%, 70%, 75%, 80%, 85%, 90%, 100%, 110%, 120%, 130%, 140%, 150%, 160%, 170%, 180%, 190%, 200%, 300%, 400%, or 500% at three months compared to the initial fluorescence at the time of implantation. In an aspect, the encasement material 109 may prevent an increase of fluorescence of more than about 60% at three months compared to the time of implantation. In some aspects, the encasement material 109 may prevent an increase of fluorescence of more than about 10%, 20%, 30%, 40%, 50%, 55%, 60%, 65%, 70%, 75%, 80%, 85%, 90%, 100%, 110%, 120%, 130%, 140%, 150%, 160%, 170%, 180%, 190%, 200%, 300%, 400%, or 500% at one year compared to the initial fluorescence at the time of implantation. In an aspect, the encasement material 109 may prevent an increase of fluorescence of more than about 150% at one year compared to the time of implantation.

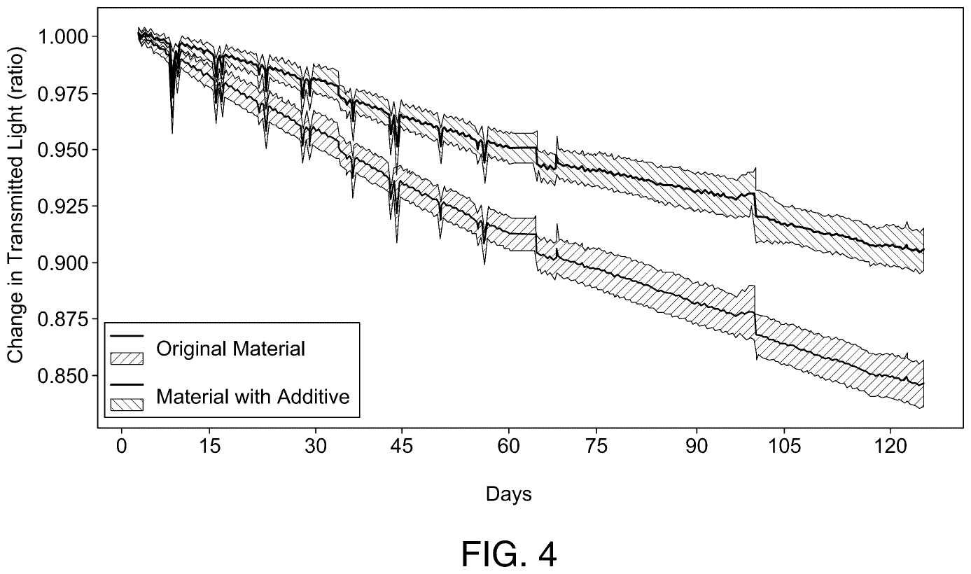

FIG. 4 presents experimental data in which encasement material 109 with the one or more additives and encasement material without the one or more additives were evaluated for the loss in transmission efficiency over time. Each encasement material's 109 transmission efficiency was normalized (i.e. transmissivity=1.000) to each material's respective initial transmissivity (i.e. day 0). As shown in FIG. 4, transmissivity declined more rapidly in the encasement material 109 without the one or more additives than in the encasement material 109 with additives. After 120 days, the encasement material 109 with the one or more additives retained at least 90% transmissivity compared to the initial transmissivity. This data can be extrapolated to predict a retention of at least 75% transmissivity compared to the initial transmissivity at one year.

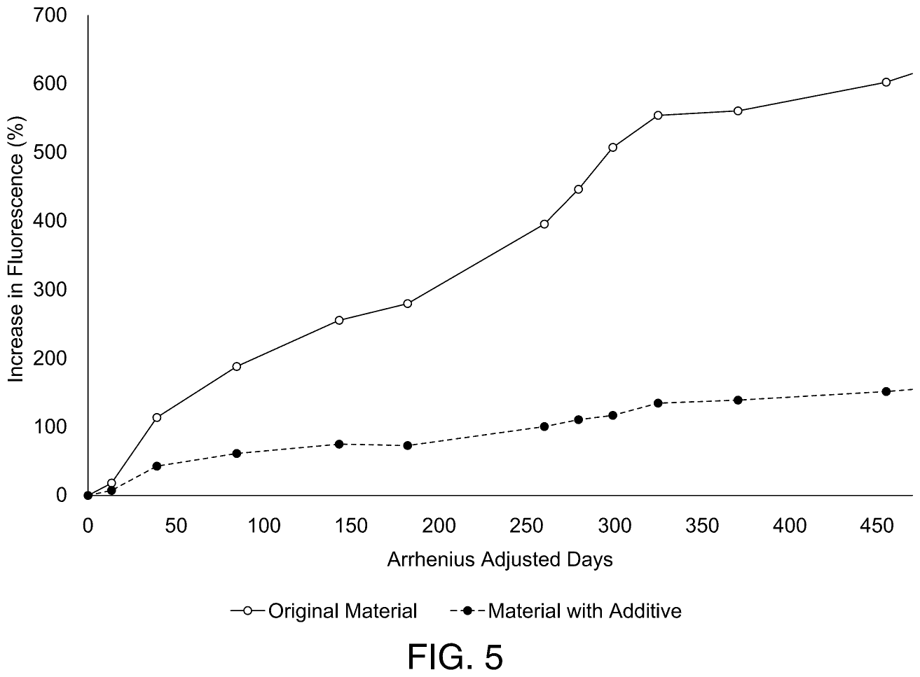

FIG. 5 presents experimental data in which encasement material 109 with the one or more additives and encasement material 109 without the one or more additives were evaluated for increased fluorescence over time. As shown in FIG. 5, fluorescence increased more rapidly in the encasement material 109 without additives than in the encasement material 109 with one or more additives. After three months (120 days), the encasement material 109 with one or more additives increased less than 60% compared to the initial fluorescence (Day 0). Also, the encasement material 109 with one or more additives increased less than 150% compared to the initial fluorescence (Day 0).

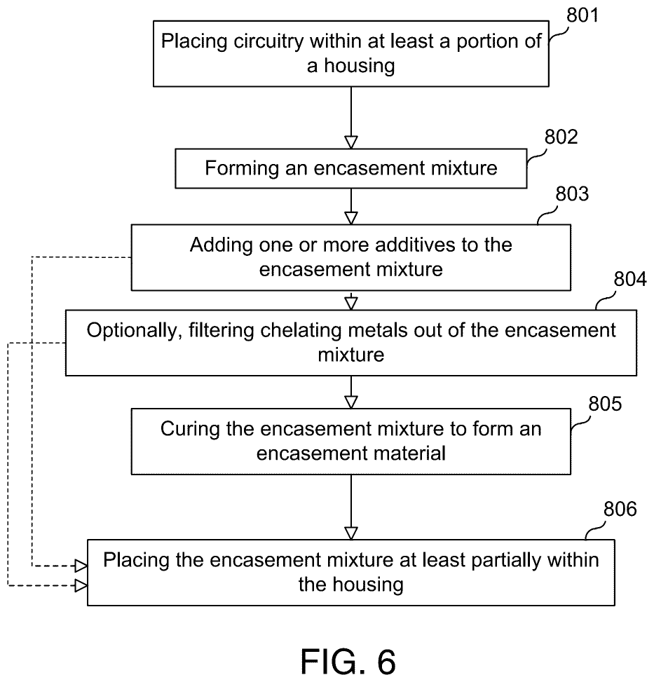

FIG. 6 illustrates a method 800 for manufacturing an apparatus 100 according to some alternative aspects. In some aspects, the method 800 may include a step 801 of placing circuitry 270 within at least a portion of a housing 102. In some aspects, the method may include a step 802 of forming an encasement mixture. In some aspects, the method 800 may include a step 803 of adding one or more additives to the encasement mixture. In some aspects, the additives may be molecules that have the ability to chelate metal ions present in the encasement mixture. In some aspects, the method 800 may include a step 804 of removing these chelated metal ions or other undesired substances from the mixture through filtering the mixture or other purification means. In some aspects, the method 800 may include a step 805 of curing the mixture so that it hardens into an encasement material 109. In some aspect, the method 800 may include a step 806 of placing the encasement material at least partially within the housing 102. In some aspects, step 806 of the method 800 may be carried out before step 805, so that the encasement mixture is placed within the housing 102 before the encasement mixture is cured to form the encasement material 109.

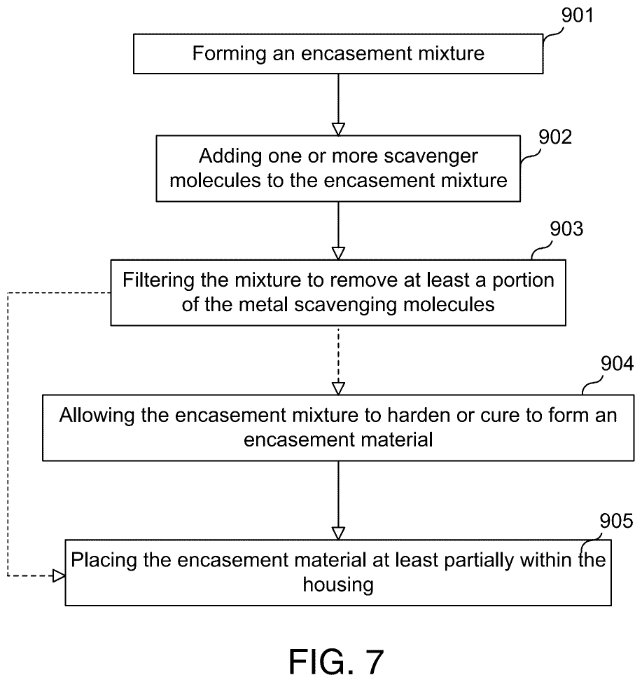

FIG. 7 illustrates a method 900 for manufacturing an apparatus 100 according to some alternative aspects. In some aspects, the method 900 may include a step 901 of forming an encasement mixture. In some aspects, the method 900 may include a step 902 of adding one or more scavenger molecules to the encasement mixture. In some aspects, the method 900 may include a step of 903 of filtering the mixture to remove at least a portion of the metal scavenging molecules. In some aspects, the method 900 may include a step of 904 of allowing the encasement mixture to harden or cure to form an encasement material. In some aspects, the method 900 may include a step of 905 of placing an encasement material at least partially within a housing 102. In some alternative aspects, the step 905 of the method 900 may be carried out before step 904, so that the encasement mixture is placed within the housing 102 before the encasement mixture is cured to form the encasement material 109.

In some aspects, the apparatus 100 may be manufactured or used through different methods. In some aspects, the method includes placing circuitry 270 at least partial within a housing 102, and placing an encasement material 109 at least partially within the housing 102. In some aspects, the encasement material 109 may include one or more additives. In some aspects, the method further includes a step that before using the encasement material 109 to encase at least the portion of the circuitry 270, metal scavenger molecules are added to the encasement material 109, followed filtering the encasement material 109 to remove metal scavenger molecules

In some aspects, the method includes using an analyte indicator 104 that covers at least a portion of an exterior surface of a housing 102 to emit an amount of emission light that is indicative of an amount or concentration of an analyte in proximity to the analyte indicator 104 and using a photodetector 224 to detect the emission light that reaches the photodetector 224. In some aspects, the circuitry 270 may include the photodetector 224. In some aspects, the circuitry 270 may be at least partially within the housing 102. In some aspects, the encasement material 109 may be at least partially within the housing 102. In some aspects, the encasement material 109 may include one or more additives configured to inhibit a reduction in transmissivity of the encasement material 109. In some aspects, the emission light 331 may reach the photodetector 224 after passing through the encasement material 109.

In some aspects, the method may include adding one or more additives to the encasement material formulation and curing the encasement material formulation to form the encasement material 109. In some aspects, no additives may be added to the encasement material formulation prior to curing to form the encasement material 109. In some aspects, the one or more additives may be mixed directly into the encasement material mixture. In some aspects, the one or more additives may be included in separate components of the epoxy mixture before setting the epoxy.

In some aspects, the one or more additives may reduce thermo-oxidation reactions, including generating peroxy radicals, dehydration reactions, carbonyl, amides, metal ligand interactions, and other oxidative reactions in the encasement material, thereby reducing chemical and spectral changes in the encasement material. In some aspects, the one or more additives may reduce thermos-oxidative reactions by performing functions, such as scavenging peroxy radicals, chelating transition metals, scavenging hydroperoxides or other reactive chemical species, inhibition of photo-oxidation, inhibition of metal-ligand interactions, inhibition of metal catalyzed oxidation, deactivation of metals, and synergistic effects with other components of the encasement material 109.

In some aspects, additives may be added to the encasement mixture before curing. In some aspects, the additives may have the ability to chelate catalytic metals. In some aspects, the catalytic metal ions that are chelated by one or more additives may be filtered out of the mixture before curing. In certain aspects, filtering the encasement mixture may remove a certain percentage of catalytic metals prior to curing. In certain aspect, filtering the encasement mixture may remove at least about 10%, 20%, 30%, 40%, 50%, 60%, 70%, 80%, 90%, 91%, 92%, 93%, 94%, 95%, 96%, 97%, 98%, 99%, 99.1%, 99.2%, 99.3%, 99.4%, 99.6%, 99.7%, 99.8%, 99.9%, or 100% of the catalytic metals prior to curing.

Aspects of the present invention have been fully described above with reference to the figures. Although the invention has been described based upon these aspects, it would be apparent to those of skill in the art that certain modifications, variations, and alternative constructions could be made to the described aspects within the spirit and scope of the invention.

### Summary of Embodiments

A1. An apparatus comprising: a housing; circuitry at least partially within the housing; and an encasement material at least partially within the housing, wherein the encasement material comprises one or more additives configured to inhibit a reduction in transmissivity of the encasement material over time.

A2. The apparatus of embodiment A1, wherein the one or more additives are configured to, in inhibiting the reduction in the transmissivity of the encasement material over time, inhibit oxidation of the encasement material.

A3. The apparatus of embodiment A1 or A2, wherein the one or more additives are configured to, in inhibiting the reduction in the transmissivity of the encasement material, inhibit formation of colored compounds in the encasement material.

A4. The apparatus of any one of embodiments A1-A3, wherein the one or more additives are further configured to inhibit formation of fluorescent compounds in the encasement material.

A5. The apparatus of any one of embodiment A1-A4, wherein the one or more additives comprise one or more antioxidant compounds.

A6. The apparatus of any one of embodiments A1-A5, wherein the one or more antioxidant compounds comprise hindered phenols, hindered amine light stabilizers (HALS), thioethers, phosphites, and/or metal deactivators.

A7. The apparatus of embodiment A5 or A6, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

A8. The apparatus of any one of embodiments A1-A7, wherein the encasement material further comprises a fully aliphatic system.

A9. An apparatus comprising: a housing; circuitry at least partially within the housing; and an encasement material at least partially within the housing, wherein the encasement material comprises a fully aliphatic system.

A10. The apparatus of embodiment A8 or A9, wherein the fully aliphatic system comprises 3,4-epoxycyclohexylmethyl 3,4-epoxycyclohexanecarboxylate and 3-aminomethyl-3,5,5-trimethylcyclohexylamine.

A11. The apparatus of any one of embodiment A8-A10, wherein the fully aliphatic system is configured to, in inhibiting the reduction in the transmissivity of the encasement material, inhibit oxidation of the encasement material.

A12. The apparatus of any one of embodiments A8-A11, wherein the fully aliphatic system is configured to, in inhibiting the reduction in the transmissivity of the encasement material, inhibit formation of colored compounds in the encasement material.

A13. The apparatus of any one of embodiments A8-A12, wherein the fully aliphatic system is further configured to inhibit formation of fluorescent compounds in the encasement material.

A14. The apparatus of any one of embodiments A1-A13, further comprising an analyte indicator that covers at least a portion of an exterior surface of the housing, wherein: the analyte indicator is configured to emit an amount of emission light that is indicative of an amount or concentration of an analyte in proximity to the analyte indicator; and the circuitry comprises a photodetector configured to detect the emission light that reaches the photodetector after passing through the encasement material.

A15. The apparatus of embodiment A14, wherein the circuitry comprises a light source configured to emit excitation light that reaches the analyte indicator after passing through the encasement material, and the analyte indicator is configured to emit the emission light in response to receiving the excitation light.

A16. The apparatus of embodiment A15, wherein the encasement material passes at least 90% of the excitation light.

A17. The apparatus of any one of embodiments A14-A16, wherein the encasement material passes at least 90% of the emission light.

A18. The apparatus of any one of embodiments A1-A17, wherein the circuitry comprises a power source.

A19. The apparatus of any one of embodiments A1-A18, wherein the circuitry comprises an antenna configured to receive power and/or communicate data.

A20. The apparatus of any one of embodiments A1-A19, wherein the encasement material adheres to the circuitry.

A21. The apparatus of any one of embodiments A1-A20, wherein the encasement material adheres to the housing.

A22. The apparatus of any one of embodiments A1-A21, wherein the housing comprises a polymer of methyl methacrylate (PMMA).

A23. The apparatus of any one of embodiments A1-A22, wherein the encasement material comprises an epoxy.

A24. The apparatus of any one of embodiments A1-A23, wherein the encasement material has an initial transmissivity at a time of implant of the apparatus, and the encasement material retains at least 90% of the initial transmissivity at 120 days following the time of implant.

A25. The apparatus of any one of embodiments A1-A24, wherein the encasement material has an initial transmissivity at a time of implant of the apparatus, and the encasement material retains at least 75% of the initial transmissivity at one year following the time of implant.

A26. The apparatus of any one of embodiments A1-A25, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

A27. The apparatus of any one of embodiments A1-A26, wherein fluorescent compounds of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following the time of implant.

A28. The apparatus of any one of embodiments A1-A27, wherein the encasement material encases at least a portion of the circuitry.

B1. A method of manufacturing an apparatus, the method comprising: placing circuitry at least partially within a housing of the apparatus; and placing an encasement material at least partially within the housing, wherein the encasement material comprises one or more additives configured to inhibit a reduction in transmissivity of the encasement material.

B2. The method of embodiment B1, wherein the one or more additives are configured to, in inhibiting the reduction in the transmissivity of the encasement material, inhibit oxidation of the encasement material.

B3. The method of embodiment B1 or B2, wherein the one or more additives are configured to, in inhibiting the reduction in the transmissivity of the encasement material, inhibit formation of colored compounds in the encasement material.

B4. The method of any one of embodiments B1-B3, wherein the one or more additives are further configured to inhibit formation of fluorescent compounds in the encasement material.

B5. The method of any one of embodiments B1-B4, wherein the one or more additives comprise one or more antioxidant compounds.

B6. The method of embodiment B5, wherein the one or more antioxidant compound comprise hindered phenols, hindered amine light stabilizers (HALS), thioethers, phosphites, and/or metal deactivators.

B7. The method of embodiment B5 or B6, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

B8. The method of any one of embodiments B1-B7, wherein the encasement material further comprises a fully aliphatic system.

B9. A method of manufacturing an apparatus, the method comprising: placing circuitry at least partially within a housing of the apparatus; and placing an encasement material at least partially within the housing, wherein the encasement material comprises a fully aliphatic system.

B10. The method of embodiment B8 or B9, wherein the fully aliphatic system comprises 3,4-epoxycyclohexylmethyl 3,4-epoxycyclohexanecarboxylate and 3-aminomethyl-3,5,5-trimethylcyclohexylamine.

B11. The method of any one of embodiments B8-B10, wherein the fully aliphatic system is configured to, in inhibiting the reduction in the transmissivity of the encasement material, inhibit oxidation of the encasement material.

B12. The method of any one of embodiments B8-B11, wherein the fully aliphatic system is configured to, in inhibiting the reduction in the transmissivity of the encasement material, inhibit formation of colored compounds in the encasement material.

B13. The method of any one of embodiments B8-B12, wherein the fully aliphatic system is further configured to inhibit formation of fluorescent compounds in the encasement material.

B14. The method of any one of embodiments B1-B13, further comprising, before using the encasement material to encase at least the portion of the circuitry, adding metal scavenger molecules to the encasement material and then filtering the encasement material to remove metal scavenger molecules.

B15. The method of any one of embodiments B1-B14, wherein the apparatus further comprises an analyte indicator that covers at least a portion of an exterior surface of the housing, wherein: the analyte indicator is configured to emit an amount of emission light that is indicative of an amount or concentration of an analyte in proximity to the analyte indicator; and the circuitry comprises a photodetector configured to detect the emission light that reaches the photodetector after passing through the encasement material.

B16. The method of embodiment B15, wherein the circuitry comprises a light source configured to emit excitation light that reaches the analyte indicator after passing through the encasement material, and the analyte indicator is configured to emit the emission light in response to receiving the excitation light.

B17. The method of embodiment B16, wherein the encasement material passes at least 90% of the excitation light.

B18. The method of any one of embodiments B1-B17, wherein the encasement material has an initial transmissivity at a time of implant of the apparatus, and the encasement material retains at least 90% of the initial transmissivity at 120 days following the time of implant.

B19. The method of any one of embodiments B1-B18, wherein the encasement material has an initial transmissivity at a time of implant, and the encasement material retains at least 75% of the initial transmissivity at one year following the time of implant.

B20. The method of any one of embodiments B1-B19, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

B21. The method of any one of embodiments B1-B20, wherein fluorescent compounds of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following the time of implant.

B22. The method of any one of embodiments B1-B21, wherein placing the encasement material at least partially within the housing comprises using the encasement material to encase at least a portion of the circuitry.

C1. A method comprising: using an analyte indicator that covers at least a portion of an exterior surface of a housing of an apparatus to emit an amount of emission light that is indicative of an amount or concentration of an analyte in proximity to the analyte indicator; and using a photodetector of the apparatus to detect the emission light that reaches the photodetector, wherein circuitry comprises the photodetector, the circuitry is at least partially within the housing, an encasement material is at least partially within the housing, the encasement material comprises one or more additives configured to inhibit a reduction in transmissivity of the encasement material, and the detected emission light reaches the photodetector after passing through the encasement material.

C2. The method of embodiment C1, wherein the one or more additives are configured to, in inhibiting the reduction in the transmissivity of the encasement material, inhibit oxidation of the encasement material.

C3. The method of embodiment C1 or C2, wherein the one or more additives are configured to, in inhibiting the reduction in the transmissivity of the encasement material, inhibit formation of colored compounds in the encasement material.

C4. The method of any one of embodiments C1-C3, wherein the one or more additives are further configured to inhibit formation of fluorescent compounds in the encasement material.

C5. The method of any one of embodiments C1-C4, wherein the one or more additives comprise one or more antioxidant compounds.

C6. The method of embodiment C5, wherein the one or more anti-oxidant compound comprise hindered phenols, hindered amine light stabilizers (HALS), thioethers, phosphites, and/or metal deactivators.

C7. The method of embodiment C5 or C6, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

C8. The method of any one of embodiments C1-C7, wherein the encasement material further comprises a fully aliphatic system.

C9. A method comprising: using an analyte indicator that covers at least a portion of an exterior surface of a housing of an apparatus to emit an amount of emission light that is indicative of an amount or concentration of an analyte in proximity to the analyte indicator; and using a photodetector of the apparatus to detect the emission light that reaches the photodetector, wherein circuitry comprises the photodetector, the circuitry is at least partially within the housing, an encasement material is at least partially within the housing, the encasement material comprises a fully aliphatic system, and the detected emission light reaches the photodetector after passing through the encasement material.

C10. The method of embodiment C8 or C9, wherein the fully aliphatic system comprises 3,4-epoxycyclohexylmethyl 3,4-epoxycyclohexanecarboxylate and 3-aminomethyl-3,5,5-trimethylcyclohexylamine.

C11. The method of any one of embodiments C8-C10, wherein the fully aliphatic system is configured to, in inhibiting the reduction in the transmissivity of the encasement material, inhibit oxidation of the encasement material.

C12. The method of any one of embodiments C8-C11, wherein the fully aliphatic system is configured to, in inhibiting the reduction in the transmissivity of the encasement material, inhibit formation of colored compounds in the encasement material.

C13. The method of any one of embodiments C8-C12, wherein the fully aliphatic system is further configured to inhibit formation of fluorescent compounds in the encasement material.

C14. The method of any one of embodiments C1-C13, wherein the circuitry further comprises a light source, the method further comprises using the light source to emit excitation light that reaches the analyte indicator after passing through the encasement material, and the analyte indicator is configured to emit the emission light in response to receiving the excitation light.

C15. The method of any one of embodiments C1-C14, wherein the encasement material passes at least 90% of the excitation light.

C16. The method of any one of embodiments C1-C15, wherein the circuitry comprises a power source.

C17. The method of any one of embodiments C1-C16, wherein the circuitry comprises an antenna configured to receive power and/or communicate data.

C18. The method of any one of embodiments C1-C17, wherein the encasement material adheres to the circuitry.

C19. The method of any one of embodiments C1C18, wherein the encasement material adheres to the housing.

C20. The method of any one of embodiments C1-C19, wherein the housing comprises a polymer of methyl methacrylate (PMMA).

C21. The method of any one of embodiments C1-C20, wherein the encasement material comprises an epoxy.

C22. The method of any one of embodiments C1-C21, wherein the encasement material has an initial transmissivity at a time of implant of the apparatus, and the encasement material retains at least 90% of the initial transmissivity at 120 days following the time of implant.

C23. The method of any one of embodiments C1-C22, wherein the encasement material has an initial transmissivity a time of at implant of the apparatus, and the encasement material retains at least 75% of the initial transmissivity at one year following the time of implant.

C24. The method of any one of embodiments C1-C23, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

C25. The method of any one of embodiments C1-C24, wherein fluorescent compounds of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following to the time of implant.

C26. The method of any one of embodiments C1-C25, wherein the encasement material encases at least a portion of the circuitry.

D1. A method comprising: forming an encasement material mixture; adding one or more metal scavenging molecules to the encasement material mixture; filtering the encasement material mixture to remove at least a portion of the metal scavenging molecules; and allowing the encasement material mixture to harden.

D2. The method of embodiment D1, wherein the encasement material mixture comprises one or more additives configured to inhibit a reduction in transmissivity of the encasement material.

D3. The method of embodiment D2, wherein the one or more additives are configured to, in inhibiting the reduction in the transmissivity of the encasement material, inhibit oxidation of the encasement material.

D4. The method of embodiment D2 or D3, wherein the one or more additives are configured to, in inhibiting the reduction in the transmissivity of the encasement material, inhibit formation of colored compounds in the encasement material.

D5. The method of any one of embodiments D2-D4, wherein the one or more additives are further configured to inhibit formation of fluorescent compounds in the encasement material.

D6. The method of any one of embodiments D2-D5, wherein the one or more additives comprise one or more antioxidant compounds.

D7. The method of embodiments D6, wherein the one or more anti-oxidant compound comprise hindered phenols, hindered amine light stabilizers (HALS), thioethers, phosphites, and/or metal deactivators.

D8. The method of embodiment D6 or D7, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

D9. The method of any one of embodiments D1-D8, wherein the encasement material comprises a fully aliphatic system.

D10. The method of embodiment D9, wherein the fully aliphatic system comprises 3,4-epoxycyclohexylmethyl 3,4-epoxycyclohexanecarboxylate and 3-aminomethyl-3,5,5-trimethylcyclohexylamine.

D11. The method of any one of embodiments D9 or D10, wherein the fully aliphatic system is configured to, in inhibiting the reduction in the transmissivity of the encasement material, inhibit oxidation of the encasement material.

D12. The method of any one of embodiments D9-D11, wherein the fully aliphatic system is configured to, in inhibiting the reduction in the transmissivity of the encasement material, inhibit formation of colored compounds in the encasement material.

D13. The method of any one of embodiments D9-D12, wherein the fully aliphatic system is further configured to inhibit formation of fluorescent compounds in the encasement material.

D14. The method of any one of embodiments D1-D13, wherein the encasement material passes at least 90% of the excitation light.

D15. The method of any one of embodiments D1-D14, wherein the housing comprises a polymer of methyl methacrylate (PMMA).

D16. The method of any one of embodiments D1-D15, wherein the encasement material comprises an epoxy.

D17. The method of any one of embodiments D1-D16, wherein the encasement material has an initial transmissivity at a time of implant of the apparatus, and the encasement material retains at least 90% of the initial transmissivity at 120 days following the time of implant.

D18. The method of any one of embodiments D1-D17, wherein the encasement material has an initial transmissivity at a time of implant of the apparatus, and the encasement material retains at least 75% of the initial transmissivity at one year following the time of implant.

D19. The method of any one of embodiments D1-D18, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

D20. The method of any one of embodiments D1-D19, wherein fluorescent compound of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following the time of implant.

E1. A method of inhibiting a reduction in transmissivity of an encasement material over time, the method comprising: adding one or more additives to an encasement material formulation; and curing the encasement material formulation to form the encasement material.

E2. The method of embodiment E1, wherein the one or more additives are configured to, in inhibiting the reduction in the transmissivity of the encasement material, inhibit oxidation of the encasement material.

E3. The method of embodiment E1 or E2, wherein the one or more additives are configured to, in slowing the reduction in the transmissivity of the encasement material, slows formation of colored compounds in the encasement material.

E4. The method of any one of embodiments E1-E3, wherein the one or more additives are further configured to inhibit formation of fluorescent compounds in the encasement material.

E5. The method of any one of embodiments E1-E4, wherein the one or more additives comprise one or more antioxidant compounds.

E6. The method of embodiment E5, wherein the one or more antioxidant compound comprise hindered phenols, hindered amine light stabilizers (HALS), thioethers, phosphites, and/or metal deactivators.

E7. The method of embodiment E5 or E6, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

E8. The method of any one of embodiments E1-E7, wherein the encasement material further comprises a fully aliphatic system.

E9. The method of embodiment E8, wherein the fully aliphatic system comprises 3,4-epoxycyclohexylmethyl 3,4-epoxycyclohexanecarboxylate and 3-aminomethyl-3,5,5-trimethylcyclohexylamine.

E10. The method of embodiment E8 or E9, wherein the fully aliphatic system is configured to, in inhibiting the reduction in the transmissivity of the encasement material, inhibit oxidation of the encasement material.

E11. The method of any one of embodiments E8-E10, wherein the fully aliphatic system is configured to, in inhibiting the reduction in the transmissivity of the encasement material, inhibits formation of colored compounds in the encasement material.

E12. The method of any one of embodiments E8-E11, wherein the fully aliphatic system is further configured to inhibit formation of fluorescent compounds in the encasement material.

E13. The method of any one of embodiments E1-E12, wherein the encasement material passes at least 90% of the excitation light.

E14. The method of any one of embodiments E1-E13, wherein the encasement material has an initial transmissivity at a time of implant of the apparatus, and the encasement material retains at least 90% of the initial transmissivity at 120 days following the time of implant.

E15. The method of any one of embodiments E1-E14, wherein the encasement material has an initial transmissivity at a time of implant of the apparatus, and the encasement material retains at least 75% of the initial transmissivity at one year following the time of implant.

E16. The method of any one of embodiments E1-E15, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

E17. The method of any one of embodiments E1-E16, wherein fluorescent compounds of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following the time of implant.

What is claimed is:

1. An apparatus comprising:
a housing;
circuitry at least partially within the housing; and
an encasement material at least partially within the housing, wherein the encasement material comprises one or more additives configured to inhibit a reduction in transmissivity of the encasement material over time.

2. The apparatus of claim 1, wherein the one or more additives are configured to, in inhibiting the reduction in the transmissivity of the encasement material over time, inhibit oxidation of the encasement material.

3. The apparatus of claim 1, wherein the one or more additives are configured to, in inhibiting the reduction in the transmissivity of the encasement material, inhibit formation of colored compounds in the encasement material.

4. The apparatus of claim 1, wherein the one or more additives are further configured to inhibit formation of fluorescent compounds in the encasement material.

5. The apparatus of claim 1, wherein the one or more additives comprise one or more antioxidant compounds.

6. The apparatus of claim 1, wherein the one or more antioxidant compounds comprise hindered phenols, hindered amine light stabilizers (HALS), thioethers, phosphites, and/or metal deactivators.

7. The apparatus of claim 5, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

8. The apparatus of claim 1, wherein the encasement material further comprises a fully aliphatic system.

9. The apparatus of claim 8, wherein the fully aliphatic system comprises 3,4-epoxycyclohexylmethyl 3,4-epoxycyclohexanecarboxylate and 3-aminomethyl-3,5,5-trimethylcyclohexylamine.

10. The apparatus of claim 8, wherein the fully aliphatic system is configured to, in inhibiting the reduction in the transmissivity of the encasement material, inhibit oxidation of the encasement material.

11. The apparatus of claim 8, wherein the fully aliphatic system is configured to, in inhibiting the reduction in the transmissivity of the encasement material, inhibit formation of colored compounds in the encasement material.

12. The apparatus of claim 8, wherein the fully aliphatic system is further configured to inhibit formation of fluorescent compounds in the encasement material.

13. The apparatus of claim 1, further comprising an analyte indicator that covers at least a portion of an exterior surface of the housing, wherein:
the analyte indicator is configured to emit an amount of emission light that is indicative of an amount or concentration of an analyte in proximity to the analyte indicator; and
the circuitry comprises a photodetector configured to detect the emission light that reaches the photodetector after passing through the encasement material.

14. The apparatus of claim 13, wherein the circuitry comprises a light source configured to emit excitation light that reaches the analyte indicator after passing through the encasement material, and the analyte indicator is configured to emit the emission light in response to receiving the excitation light.

15. The apparatus of claim 14, wherein the encasement material passes at least 90% of the excitation light.

16. The apparatus of claim 13, wherein the encasement material passes at least 90% of the emission light.

17. The apparatus of claim 1, wherein the encasement material has an initial transmissivity at a time of implant of the apparatus, and the encasement material retains at least 90% of the initial transmissivity at 120 days following the time of implant.

18. The apparatus of claim 1, wherein the encasement material has an initial transmissivity at a time of implant of the apparatus, and the encasement material retains at least 75% of the initial transmissivity at one year following the time of implant.

19. The apparatus of claim 1, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

20. The apparatus of claim 1, wherein fluorescent compounds of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following the time of implant.

21. A method of manufacturing an apparatus, the method comprising:
placing circuitry at least partially within a housing of the apparatus;
placing an encasement material at least partially within the housing, wherein the encasement material comprises one or more additives configured to inhibit a reduction in transmissivity of the encasement material.

22. The method of claim 21, wherein the one or more additives are configured to, in inhibiting the reduction in the transmissivity of the encasement material, inhibit oxidation of the encasement material.

23. The method of claim 21, wherein the one or more additives are configured to, in inhibiting the reduction in the transmissivity of the encasement material, inhibit formation of colored compounds in the encasement material.

24. The method of claim 21, wherein the one or more additives are further configured to inhibit formation of fluorescent compounds in the encasement material.

25. The method of claim 21, wherein the one or more additives comprise one or more antioxidant compounds.

26. The method of claim 25, wherein the one or more antioxidant compound comprise hindered phenols, hindered amine light stabilizers (HALS), thioethers, phosphites, and/or metal deactivators.

27. The method of claim 25, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

28. The method of claim 21, wherein the encasement material further comprises a fully aliphatic system.

29. The method of claim 21, further comprising, before using the encasement material to encase at least the portion of the circuitry, adding metal scavenger molecules to the encasement material and then filtering the encasement material to remove metal scavenger molecules.

30. The method of claim 21, wherein the apparatus further comprises an analyte indicator that covers at least a portion of an exterior surface of the housing, wherein:
the analyte indicator is configured to emit an amount of emission light that is indicative of an amount or concentration of an analyte in proximity to the analyte indicator; and
the circuitry comprises a photodetector configured to detect the emission light that reaches the photodetector after passing through the encasement material.

31. The method of claim 30, wherein the circuitry comprises a light source configured to emit excitation light that reaches the analyte indicator after passing through the encasement material, and the analyte indicator is configured to emit the emission light in response to receiving the excitation light.

32. The method of claim 31, wherein the encasement material passes at least 90% of the excitation light.

33. The method of claim 21, wherein the encasement material has an initial transmissivity at a time of implant of the apparatus, and the encasement material retains at least 90% of the initial transmissivity at 120 days following the time of implant.

34. The method of claim 21, wherein the encasement material has an initial transmissivity at a time of implant, and the encasement material retains at least 75% of the initial transmissivity at one year following the time of implant.

35. The method of claim 21, wherein fluorescent compounds of the encasement material produce an initial amount of fluorescence at a time of implant of the apparatus, and the encasement material prevents an increase of the fluorescence produced by the fluorescent compounds of the encasement material of more than 60% of the initial amount of fluorescence at three months following the time of implant.

36. The method of claim 21, wherein fluorescent compounds of the encasement material produce an initial amount of fluorescence at a time of implant of the apparatus, and the encasement material prevents an increase of the fluorescence produced by the fluorescent compounds of the encasement material of more than 150% of the initial amount of fluorescence at one year following the time of implant.

37. A method comprising:
using an analyte indicator that covers at least a portion of an exterior surface of a housing of an apparatus to emit an amount of emission light that is indicative of an amount or concentration of an analyte in proximity to the analyte indicator; and
using a photodetector of the apparatus to detect the emission light that reaches the photodetector, wherein circuitry comprises the photodetector, the circuitry is at least partially within the housing, an encasement material is at least partially within the housing, the encasement material comprises one or more additives configured to inhibit a reduction in transmissivity of the encasement material, and the detected emission light reaches the photodetector after passing through the encasement material.

38. The method of claim 37, wherein the one or more additives are configured to, in inhibiting the reduction in the transmissivity of the encasement material, inhibit oxidation of the encasement material.

39. The method of claim 37, wherein the one or more additives are configured to, in inhibiting the reduction in the transmissivity of the encasement material, inhibit formation of colored compounds in the encasement material.

40. The method of claim 37, wherein the one or more additives are further configured to inhibit formation of fluorescent compounds in the encasement material.

41. The method of claim 37, wherein the one or more additives comprise one or more antioxidant compounds.

42. The method of claim 41, wherein the one or more anti-oxidant compound comprise hindered phenols, hindered amine light stabilizers (HALS), thioethers, phosphites, and/or metal deactivators.

43. The method of claim 41, wherein the one or more antioxidant compounds comprise octadecyl 3-(3,5-di-tert-butyl-4-hydroxyphenyl) propionate, pentaerythritol tetrakis(3,5-di-tert-butyl-4-hydroxyhydrocinnamate), 2,2′-thiodiethylene bis[3-(3,5-di-tert-butyl-4-hydroxyphenyl) propionate], didodecyl 3,3′-thiodipropionate, bis(2,2,6,6-tetramethyl-4-piperidyl) sebacate, Sumilizer AG-80, tris(2-nonylphenyl) phosphite, octyl-3,5-di-tert-butyl-4-hydroxy-hydrocinnamate, 1,1,3-tris(2-methyl-4-hydroxy-5-tert-butylphenyl) butane, 1,2-bis(3,5-di-tert-butyl-4-hydroxyhydrocinnamoyl) hydrazine, 2-hydroxy-N-(1H-1,2,4-triazol-3-yl)benzamide, and/or N′1,N′12-bis(2-hydroxybenzoyl) dodecanedihydrazide, and/or (ethane-1,2-diylbis(oxy))bis(ethane-2,1-diyl) bis(3-(3-(tert-butyl)-4-hydroxy-5-methylphenyl) propanoate).

44. The method of claim 37, wherein the encasement material further comprises a fully aliphatic system.
